(19) United States
(12) Patent Application Publication
(10) Pub. No.: US 2005/0008745 A1
(43) Pub. Date: Jan. 13, 2005

(54) Method for sterilising products

(76) Inventor: Jirka Blaha, Lorraach (DE)

Correspondence Address: WENDEROTH, LIND & PONACK, L.L.P., 2033 K STREET N. W., SUITE 800, WASHINGTON, DC, 20006-1021, US

(21) Appl. No.: 10/493,838

(22) PCT Filed: Nov. 1, 2002
(86) PCT No.: PCT/CH02/00591

(30) Foreign Application Priority Data
Nov. 2, 2001 (CH) 2007/01

(51) Int. Cl. A23L 3/16

(52) U.S. Cl. 426/521

(57) ABSTRACT

The invention relates to a method for sterilising products, especially found products and the raw materials, auxiliary products and semi-finished products thereof, in additions to raw materials, adjuvants and Intermediaries for the pharmaceutical and cosmetic industry. The products which are separated by sorting are placed, in product-dependent layer thicknesses. In a process container (3), after being possibly separated and/or sieved. The process container (3) guarantees identical treatment conditions for the entire product load, with the aim of achieving homogeneous product quality. The process container is introduced into a pressure chamber (7) in which the actual sterilisation process is controlled by a programme and runs homogeneously. The process container is removed from the pressure chamber (7), the product is then removed from the precess container, before or after being cooled to a pre-determined temperature, and is optionally brought back to its original form by means of a mechanical aftertreatment before being placed in the delivery container and/or being further processed. The invention thus enables the surface of products, especially natural dry products, to be sterilised and disinfected in an homogeneous and reproducible manner without causing any unwanted change in the sensory, physiological and chemical product properties.

The invention relates to a process for sterilising products, in particular foodstuffs and their raw materials, auxiliary products and semi-finished products, together with raw materials, adjuvants and intermediaries for the pharmaceutical and cosmetic industries, in accordance with the heading of claim 1, together with a device for carrying out the process.

Various processes and equipment for the natural sterilisation of plant, animal or synthetic materials are known, in which the product to be sterilised, preferably loaded into bags, is subjected to sterilisation, using steam and/or saturated steam, in a processing chamber which can be sealed and evacuated. From EP-A-0 998 855 it is also known that in the processing chamber one or more charging containers can be holding the product to be sterilised can be accommodated, whereby particularly the sterilisation of powdery materials is improved (shorter processing time, more even processing of the product to be sterilised).

The present invention derived from the task of creating a process of the type mentioned at the beginning, plus a device for the implementation of this process, which would facilitate particularly efficient and gentle sterilisation and, where appropriate, disinfection, whilst preserving homogeneous and largely unaltered product characteristics (quality).

This task is solved by the invention with a process with the characteristics of claim 1 and with a device with the characteristics of claim 15.

The preferred alternative versions of the invention are the subject of secondary claims. The process according to the invention and the device for carrying it out according to the invention facilitate constant and reproducible treatment of products. This results in a high degree of preservation of homogeneity, sensory properties (aroma, colour, taste, grain etc.) of the functional components and of the nutritional values (vitamins, minerals, carbohydrates, fats, protein etc.) of the products.

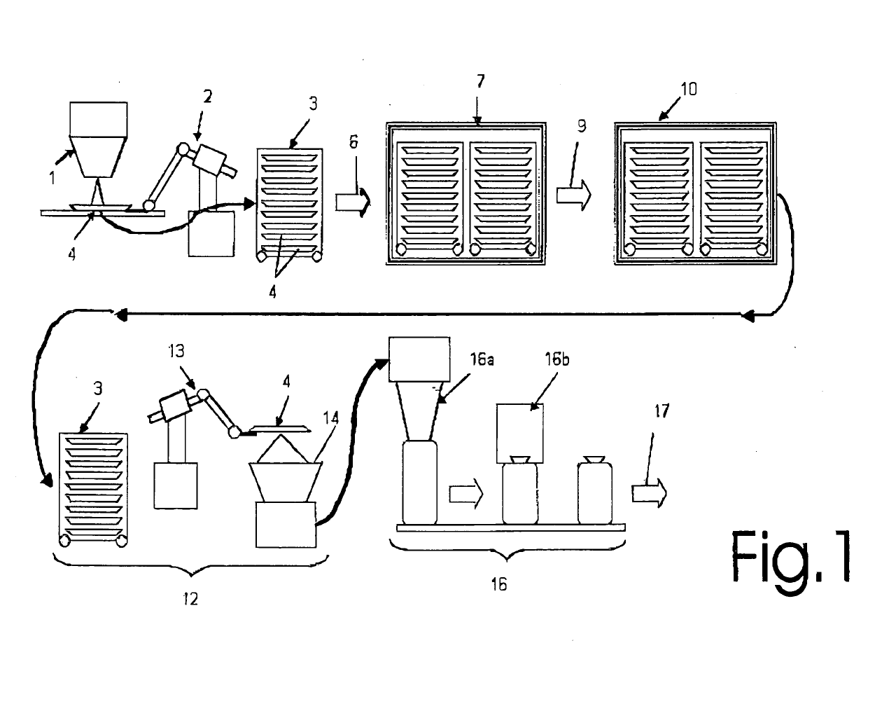

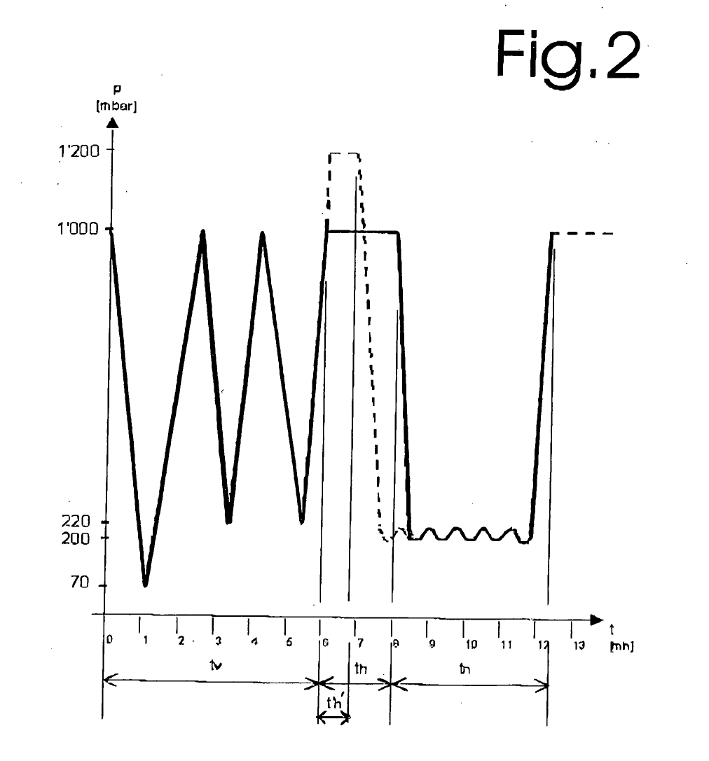

The invention is explained in more detail below with the help of FIGS. 1 and 2.

FIG. 1 Diagrammatic view of a process according to the invention and a device according to the invention; and

FIG. 2 Pressure/time diagram of a sterilisation and disinfection process according to the invention.

FIG. 1 is a diagrammatic illustration of a process and a device for sterilisation and disinfection. These are suitable for the processing of products, in particular dry products, such as foodstuffs and their raw materials, auxiliary products and semi-finished products, together with raw materials, adjuvants and intermediaries for the pharmaceutical and cosmetic industries. The products to be processed are delivered in the form of powder, granulates, flakes and in whole or cut pieces (e.g. seeds, nuts, leaves, roots, dried fruit and vegetables etc.). Various drums and/or containers (e.g. boxes, so-called big bags, sacks, silos etc.) may be used as transport containers. All the products are subjected to an incoming control. Using a reference sample the products are tested for water content, sensory properties (e.g. appearance, smell, aroma, taste etc.) and other product-specific properties. The results are recorded on a protocol. Up to the start of the process the products are stored separately for each type. Special storage regulations and guidelines concerning the general transmission of odours and the guidelines for processing bio-products have to be observed. Certain products are stored in a refrigerated environment.

The water content of the products to be treated is tested again immediately before treatment. Before treatment in the pressure and/or sterilisation chamber certain products may be subjected to one or more mechanical treatments (for example sticky products may be broken up or somewhat lumpy products may be sieved).

The actual treatment process begins by filling the product (via a funnel) into a metering device 1. With the help of the metering device 1 the products are distributed in defined layers on trays 4 as required. In particular in the case of powders, granulates or flakes the thickness of the layer is precisely observed. The trays 4 with the products to be treated are placed manually or automatically into a process container designed as a tray-holder 3. Naturally instead of the tray-holder 3 and the trays 4 other process containers and/or charging containers may be used. The process container may also be designed as a bag-holder, which ensures even vaporization of the surfaces of the bags.

As indicated in FIG. 1 with arrow 6, according to the invention the preferably moveable tray-holder or holders 3 are then pushed manually or automatically through an entry hatch into the sterilisatlon chamber 7.

In the sterilisation chamber the actual program-controlled sterilisation process takes place. This basically comprises three phases, namely:
(a) conditioning
(b) sterilisatlon and
(c) drying of the products.

The gas-tight sterilisation chamber 7 is connected to a vacuum pump, a steam supply (preferably overheated steam or saturated steam) and an air supply, though these are not shown in detail.

An example of how a sterilisatlon process conducted with saturated steam may operate in a particularly advantageous way is shown in the pressure/time diagram in FIG. 2. This shows that in the first phase in a product-specific space of time, a gas exchange and the heating of the product to be sterilised take place. This so-called conditioning phase, which runs through several vacuum cycles, allows the next so-called treatment phase to be kept short (time tₕ) and at relatively low temperatures (saturated steam temperature preferably 70°-120° C.). At the start of the treatment phase the sterilisation chamber 7 is under a deep vacuum, which ensures the rapid and complete contact of the steam with every part of the surface of the product. The absolute pressure values are preferably between 50 and 1000 mbar. For certain products the treatment may be further shortened with a temperature of more than 100° C. and an absolute pressure of over 1200 mbar (cf. the broken line in FIG. 2, time tₕ′). The so-called holding time during which a constant temperature and level of pressure are maintained should preferably be 30 s to 300 s. In these conditions gentle, high grade sterilisation is guaranteed, generally without diminishing the nutritional and/or sensory properties. The treatment phase is followed by the so-called drying phase, during which the product to be sterilised is dried and partially cooled for a product-specific time tₛ. By a pressure reduction in the vacuum area any condensation water is removed.

As already mentioned the sterilization process takes place in the sterilisation chamber 7 fully automatically and under computer control. The main process parameter during the conditioning phase is the pressure change (pressure, number and duration of vacuum cycles, temperature and speed of gas exchange and pressure reduction). During the treatment phase, the actual sterilisation process, it is the holding time, pressure and temperature which constitute the main factors. In the after-treatment and/or drying it is the duration, pressure pattern, number of air blasts, air quality and initial and end temperatures.

After completion of the sterilisation process the tray-holder or holders 3 containing the products are removed from the sterilisation chamber 7. This takes place e.g. through an exit hatch opposite the entry hatch. The product is then introduced into a cooling chamber 10 in the direction of the arrow 9. At this point the temperature on the surface of the product is as a rule 50 to 60° C. The air in the cooling chamber 10 is conditioned in terms of temperature, humidity and cleanliness. For the cooling conditions product-specific criteria are set. The main process parameters of the likewise program-controlled cooling phase are the initial and end temperatures and the duration of the cooling process.

The trays 4 with the products cooled to the preset temperature are removed from the tray holders 3 in an emptying station 12 manually or automatically in a manipulator or similar and emptied into an emptying funnel 14.

Products which form lumps or stick together during the sterilisation process are treated mechanically before and/or after cooling (ground, granulated, separated) in order to restore them to their initial consistency. Vibrating screens, nibblers or other units may be used as separating devices. If the products are mechanically processed before cooling, the cooling takes place in a suitable process container.

Using a packing device 16 the products are then filled into the product-specific outgoing delivery recipients or delivery containers (cf. supply reference 16a). These recipients may be designed in as many diverse forms as there are incoming delivery containers. Immediately before sealing these recipients (cf. supply reference 16b) a reference sample is taken.

The process steps 9-16 take place preferably in spaces where the air has been conditioned in terms of temperature, relative humidity and microbial load (so-called clean room).

The sealing and transporting of the outgoing delivery recipients completes the treatment process (arrow 17 indicates the onward transfer from the clean room).

Where appropriate the process container may also be designed to be suitable for transferring the treated products on to a further product processing process.

The process as per the invention described above makes it possible to sterilise products in an efficient and gentle way, in particular dry products, thus to a large degree ensuring preservation of the homogeneity, sensory properties (aroma, colour, taste, grain etc.), functional component substances and nutritional values (vitamins, minerals, carbohydrates, fats, protein etc,) of the products. One important advantage is that the treatment is reproducible and leads to a homogeneous quality of the product batch.

1-17. (Cancelled)

18. Process for the sterilisation of products, In particular foodstuffs and their raw materials, auxiliary products and semi-finished products, and also raw materials, adjuvants and intermediaries for the pharmaceutical and cosmetic industries, wherein the products, sorted according to type, and if necessary separated and/or sieved, are distributed in layers whose thickness depends on the product, in a process container, whereby the process container guarantees identical treatment conditions for the whole product batch with a view to achieving homogeneous product quality, whereupon the process container is inserted into a pressure chamber in which the actual sterilisation process takes place In a program-controlled and homogeneous manner, whereafter the process container is removed from the pressure chamber and the product is taken out of the process container before or after it is cooled to a preset temperature, in order then to be restored to its original form by means of mechanical after-treatment if necessary, before it is then filled into the outgoing delivery container and/or forwarded for further processing.

19. Process according to claim 18, wherein the product surface of natural dry products is sterilised and disinfected homogeneously and reproducibly with no unwanted alteration of the sensory, physiological and chemical product properties.

20. Process according to claim 18, wherein the products to be sterilised and disinfected are distributed in preset layer thicknesses on trays by means of a metering device and accommodated in a process container designed as a tray-holder.

21. Process according to claim 18, wherein the products to be treated and disinfected are distributed in steam permeable bags in product-dependent layer thicknesses and that these are then accommodated in a process container designed as a bag-holder.

22. Process according to claim 18, wherein the product is restored to its original form after cooling by means of a suitable tool (preferably a nibbler).

23. Process according to claim 18, wherein the saturated steam is used to implement the sterilisation process.

24. Process according to claim 23, wherein the saturated steam is supplied as pharmaceutical pure steam.

25. Process according to claim 18, wherein the actual sterilisation and disinfection process is carried out in three phases, namely conditioning, sterilisation and disinfection, together with drying.

26. Process according to claim 23, wherein the pressure chamber is placed under a vacuum in several cycles for the conditioning and the drying.

27. Process according to claim 23, wherein the fact that the sterilisation and disinfection take place during a period of preferably 30 to 300 s and at temperatures of between 700 and 120° C., whereby steam input starts under negative pressure.

28. Process according to claim 18, wherein after leaving the pressure chamber (7) up to and including filling into the outgoing delivery containers, the sterilised and disinfected products do not leave a clean room, with the exception of products which are sterilised and disinfected directly in the outgoing delivery containers.

29. Process according to claim 28, wherein the air is conditioned in a clean room in terms of temperature, relative humidity and microbial load, according to the product.

30. Process according to claim 18, wherein the products are cooled passively by leaving them in the ambient air and/or actively in a cooling chamber.

31. Process according to claim 18, wherein the outgoing delivery container displays a permeability to air and water specific to the product and allows no microbial recontamination.

32. Device for the execution of the process according to claim 18, wherein a metering device (1), a device (2) for introducing the products into a process container (3), a pressure chamber (7), a cooling chamber (10), an emptying device (12) for emptying the process container, devices for sieving, grinding, breaking up and separating products, a packing device (16), control devices for determining the temperature, water content and water availability in and around the product together with control units for controlling the sterilisation and disinfection process and cooling.

33. Device according to claim 32, wherein the process container is preferably designed as a moveable tray-holder, into which trays with layers of the product on can be inserted.

34. Device according to claim 32, wherein the process container is designed as a moveable bag-holder, which ensures even vaporisation of the surfaces of the bag.
